(19) United States
(12) Patent Application Publication
(10) Pub. No.: US 2005/0251226 A1
(43) Pub. Date: Nov. 10, 2005

(54) Suppression of tinnitus

(76) Inventor: John P. D'Angelo, Medford, MA (US)

Correspondence Address: John P. D'Angelo, 30 Sherman Place, Medford, MA, 02155, US

(21) Appl. No.: 11/125,949

(22) Filed: May 9, 2005

Related U.S. Application Data
(60) Provisional application No. 60/568,920, filed on May 7, 2004.

(51) Int. Cl. A61N 1/18

(52) U.S. Cl. 607/57

(57) ABSTRACT

The perception of sound in the absence of a stimulus external to one's own ear is commonly referred to as “tinnitus”. However, other sounds such as otoacoustic emissions, may occur in the absence of an external stimulus. These sounds may be audible or inaudible. A means of reducing such sounds is presented. For the audible case, the device first measures the sound and then injects a sound of proper frequency content thus canceling the tinnitus or otoacoustic emission. The inaudible case requires other means of sensing the perceived sound. Suppression of the noise perceived by one suffering from inaudible tinnitus is outlined. Such a response may be processed similar to the microphone signal acquired for the audible case. Sound of proper frequency content is then injected into the affected ear and the neurological response due to the generated sound would then cancel the inaudible tinnitus.

### CROSS-REFERENCE TO RELATED APPLICATIONS

This application claims priority to U.S. provisional patent application No. 60/568,920, filed May 7, 2004, the contents of which are incorporated in their entirety.

### BACKGROUND

Any and all sounds—audible or inaudible, measurable or immeasurable—perceived to exist but without the benefit of an external stimulus, including but not limited to tinnitus and various forms of otoacoustic emissions including spontaneous otoacoustic emissions, evoked otoacoustic emissions and delayed evoked otoacoustic emissions, are referred to herein collectively as “tinnitus”. Tinnitus which is capable of being measured using a microphone is referred to herein as “audible tinnitus”. Tinnitus which is not capable of being measured, but still is perceived by a patient to exist, is referred to herein as “inaudible tinnitus”.

The invention generally relates to a means by which the affects of tinnitus may be suppressed or even abolished by injecting sound of proper frequency content with proper amplitude and phase into the affected ear. Such a system would have a means to sense the tinnitus, account for the acoustic effects of the ear and generate an audible signal to suppress said tinnitus.

The use of noise suppression techniques is generally known to those skilled in the art and has been used successfully in various acoustics problems having very complex acoustic fields. However, such concepts have not been explored for the suppression of tinnitus.

Tinnitus is common; estimates of its prevalence range up to 80 percent of all adults. About ten percent of adults complain of chronic tinnitus, and about 0.5% of all adults describe their tinnitus as interfering with their ability to lead a normal life. Tinnitus may have many different etiologies but in about 50% of cases no etiology can be determined and may occur in either or both of a patient's ears. Tinnitus has also been determined to be both audible and inaudible. Although audible and inaudible tinnitus each are perceived to exist by the patient they may not both be measured using a microphone placed in the ear canal. Audible tinnitus may be detected with a stethoscope or a low-noise microphone placed within the ear canal and, in extreme cases, may be heard close to the patient's ear by others whereas for inaudible tinnitus no acoustic signal may be detected.

### SUMMARY OF THE INVENTION

Stated herein is an invention which intends to identify and reduce the magnitude of a sound perceived by a patient having either audible or inaudible tinnitus. References are made to the diagram of the human ear shown in FIG. 1.

Knowledge of the acoustic relationship between the ear canal and the cochlea is important. The device envisioned to be used to measure a signal correlated with the tinnitus—such as but not limited to a microphone—and be capable of generating a secondary acoustic field will be located, in whole or in part, within the ear canal. However, it is the acoustic pressure occurring in the cochlea, which is ultimately heard by the patient. Therefore, knowledge of such acoustic relations is beneficial. The relation of acoustic pressure existing in the middle ear and at the cochlea is referred to as the middle ear acoustic transfer function. This acoustic relationship is nonlinear being dependent on the direction that the acoustic pressure wave is traveling. FIG. 2 shows the forward—from the ear canal to the cochlea—and the reverse—from the cochlea to the ear canal—middle ear acoustic transfer functions are frequency dependent and are referred to as M1 and M2, respectively.

The device, once sensing the tinnitus, then processes the information and generates a secondary acoustic field within the ear canal. The acoustic characteristics of the secondary acoustic field are modified via the forward middle ear acoustic transfer function (M1) such that at the cochlea the secondary acoustic field is of proper frequency content, phase and amplitude to minimize the tinnitus perceived by the patient.

### BRIEF DESCRIPTION OF THE DRAWINGS

These and other features, aspects, and advantages of the present invention will become better understood with regard to the following description, appended claims, and accompanying drawings where:

FIG. 1 shows the location of a device placed in the ear canal and used for the suppression of tinnitus.

FIG. 2a shows the components and the general design of the device used for the suppression of tinnitus.

FIG. 2b shows the components and the alternative design of the device used for the suppression of tinnitus.

FIG. 3a shows, in schematic form, the forward and reverse middle ear pressure gains.

FIG. 3b shows the forward and reverse middle ear pressure gains in relation to the pertinent components of the human ear.

FIG. 4 shows a schematic illustrating the indirect suppression of audible tinnitus.

FIG. 5 shows a schematic illustrating the direct suppression of audible tinnitus.

FIG. 6 shows a schematic illustrating the suppression of inaudible tinnitus.

FIG. 7 shows a schematic illustrating the suppression of inaudible tinnitus using manual tunings.

### DETAILED DESCRIPTION OF THE INVENTION

The present invention is a device directed to reducing sound heard or perceived by an individual suffering from tinnitus. The device is designed to fit within the ear canal, either in whole or in part, of the ear in which the tinnitus is heard or perceived.

The device of the present invention is capable of generating acoustic energy—both tonal and broadband—over a wide range of frequencies. Such acoustic energy is generated such that the real or perceived sound due to tinnitus may be suppressed.

The present invention utilizes at least one sensor, such as but not limited to a microphone, capable of measuring a signal correlated with the tinnitus either heard or perceived by the patient; at least one acoustic source, such as but not limited to at least one loud speaker; at least one control system along with necessary analog filtering and amplification components; and a source of power, such as but not limited to the use of at least one battery.

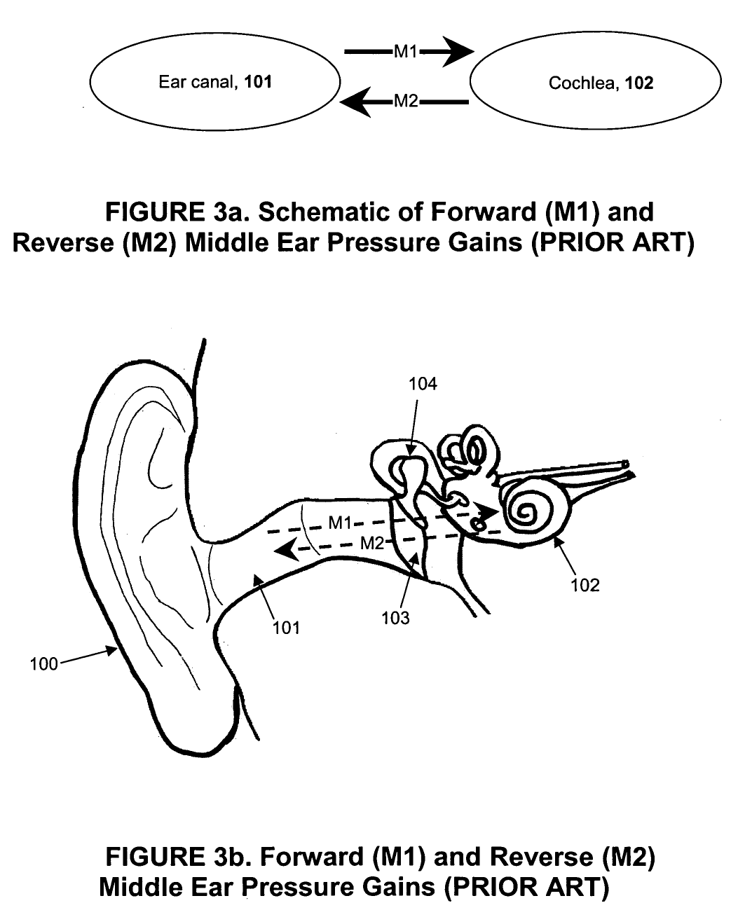

The indirect minimization method involves suppressing a real, measurable acoustic pressure due to tinnitus in the ear canal at the point of measurement. This control approach requires the controller have knowledge of M1 and M2 or some other acoustic relations between the ear canal and the cochlea. However, the resulting acoustic pressure at the cochlea may then be estimated knowing the total pressure in the ear canal and the estimated M1 as depicted in the diagram shown in FIG. 3.

Acoustic pressure due to tinnitus is sensed in the ear canal, pₑ₁. This sensed acoustic pressure may then be minimized, at the point of measurement, within the ear canal using a collocated or nearly-collocated sensor/source pair such as but not limited to a microphone and loudspeaker. The control system generates a secondary acoustic pressure, pₑ₂, being of equal frequency content and magnitude and opposing phase relative to the primary acoustic pressure, pₑ₁. The secondary acoustic pressure wave then cancels or suppresses the primary acoustic pressure wave due to the tinnitus such that |pₑ₁+pₑ₂|<|pₑ₁|. The suppression perceived by the patient is then a by-product of the control effort performed to minimize the acoustic pressure due to tinnitus occurring in the ear canal at the point of measurement. This suppression would then be the acoustic pressure at the cochlea due to the tinnitus, p_{c1}, summed with the acoustic pressure resulting at the cochlea p_{c2}, due to the generated acoustic pressure in the ear canal, pₑ₂, convolved with the actual forward middle ear acoustic transfer function, M1, such that |p_{c1}+_{pc2}|<|p_{c1}|. Note, however, for the indirect minimization of audible tinnitus, it is emphasized that the pressures at the cochlea are not considered in the controller W(f).

The direct minimization method also involves the suppression of audible tinnitus but first requires estimating the acoustic relation between the ear canal at or near the point of measurement and the cochlea. Such an estimate may be made using, but not limited to, the middle ear acoustic transfer functions M1 and M2 as described above.

The direct minimization method uses a control system having benefit either of previously determined knowledge of the acoustic relation between the ear canal and the cochlea or the ability to estimate such a relation prior to and/or during the control effort. Acoustic pressure originating at the cochlea, p_{c1}, convolved with the actual reverse middle ear acoustic transfer function, M2, results in an acoustic pressure in the ear canal, pₑ₁, due to the audible tinnitus. The acoustic pressure in the ear canal, pₑ₁, is measured by the device and processed by the controller, W(f), which estimates p_{c1} and generates a secondary acoustic pressure in the ear canal, pₑ₂ which intends to result in p_{c2} when convolved with the actual forward middle ear acoustic transfer function, M1. The two pressures at the cochlea, p_{c1} and p_{c2}, are of equal magnitude and frequency content but of opposing phase thus suppressing the level of tinnitus perceived by the patient such that |p_{c1}+p_{c2}|<|p_{c1}|.

This method is based on existing knowledge of the operations of the ear and the mechanisms by which tinnitus is perceived. Further, it assumes that all noise measured in the ear canal originates from the cochlea. However, this may not be the case since the condition causing the tinnitus may be directly contributing to the ear canal recording, such as through bone conducted pathways directly to the ear canal. Therefore, to take full advantage of the direct method may require further advances in the ability to predict the cochlea response from the ear canal measurements.

In the case of the minimization of inaudible tinnitus, no acoustic pressure may be measured in the ear canal although the patient does indeed perceive a disturbance in the absence of any noise source external to the affected ear. It is conceived that a signal, correlated with the perceived disturbance due to the inaudible tinnitus, may be sensed, either directly or indirectly, by measuring, for instance but not limited to, the activity of the nervous system. Such a measurement may be used to identify the frequency content, magnitude and phase of the perceived sound such that a real, measurable acoustic pressure may be generated in the ear canal. Such a sound may be generated such that it effectively cancels the perceived noise due to the inaudible tinnitus. In other words, an acoustic pressure may be generated to cancel the perceived noise due to the inaudible tinnitus.

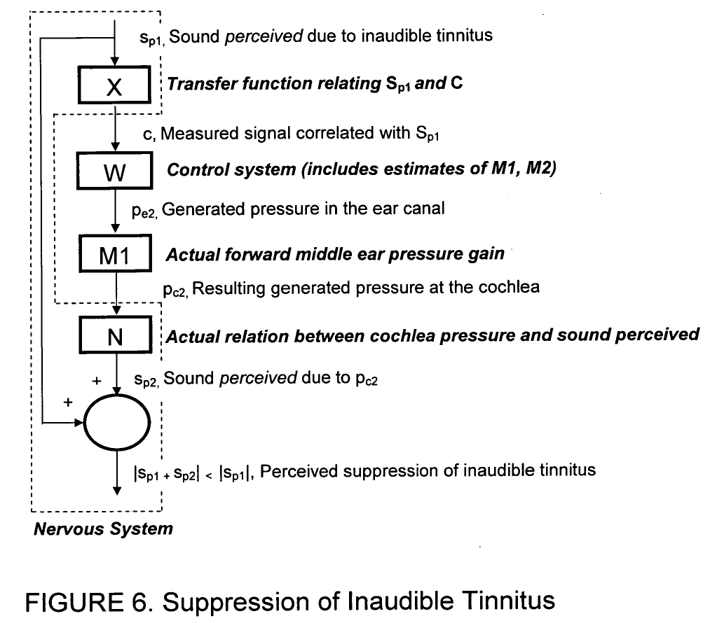

FIG. 6 shows this innovation. A signal, C, correlated with the sound, sₚ₁, perceived by the subject due to the inaudible tinnitus, is sensed. Such a signal, c, may be, but is not limited to be, that of measured neurological activity. The signal, c, may then be input to the controller, W(f), such that a measurable acoustic pressure, pₑ₂, may then be generated in the ear canal which, via M1, results in an acoustic pressure existing at the cochlea, p_{c2}. This real, measurable acoustic pressure existing in the cochlea may then be perceived as sₚ₂ by the subject being of equal magnitude and frequency content yet of opposite phase to that of the perceived signal due to the inaudible tinnitus, sₚ₁. Therefore, the two perceived sounds—the first due to the inaudible tinnitus and the second due to the acoustic pressure generated by the controller—then cancel resulting in a perceived suppression of the inaudible tinnitus such that |sₚ₁+sₚ₂|<|sₚ₁|.

This process may also be accomplished by generating an acoustic pressure within the ear canal in the absence of any measured knowledge of the inaudible tinnitus. In this instance, suppression is achieved by generating such a pressure and iteratively varying the frequency content and phase until the patient perceives a reduction of the disturbance.

The dimensions of the components of the acoustic treatment described within the present invention specified herein are for exemplary purposes illustrating the details of one particular configuration. These dimensions may vary depending on the application and are not to be considered limitations of the present invention.

The acoustic treatment of the present invention uses at least one device placed either in whole or in part within the affect ear canal, consisting of at least one sensor, such as but not limited to a microphone, capable of sensing a response correlated with the perceived tinnitus, at least one acoustic source, such as but not limited to an ordinary loud speaker, and at least one controller capable of processing the recorded information and generating the appropriate input to the at least one noise source such that the perceived tinnitus may be suppressed. It is envisioned that this device may be used in either ear simultaneously for cases of bilateral tinnitus—those cases where tinnitus is perceived in both ears.

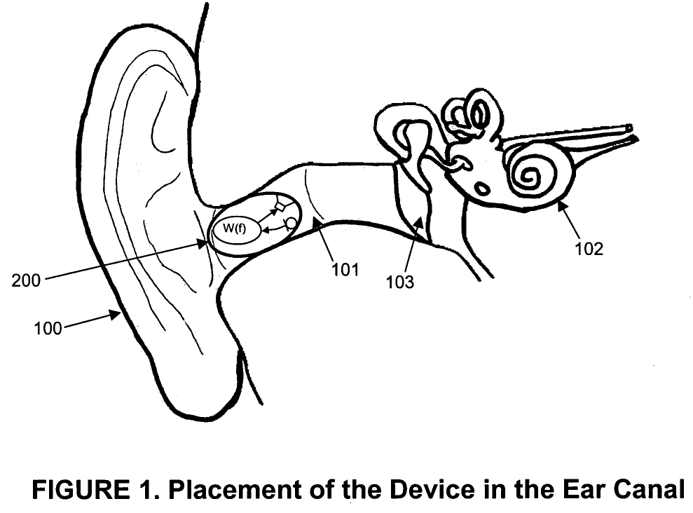

In FIG. 1, the cross section of a human ear 100 is shown. A device 200 is shown placed within the ear canal 101. For this invention it is of particular interest to note the cochlea 102 and ear drum (tympanic membrane) 103.

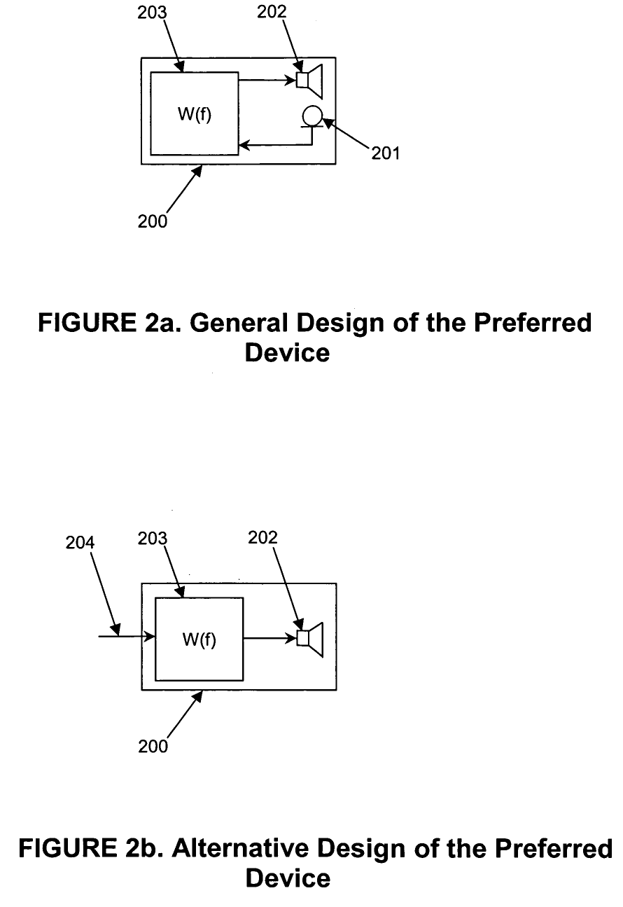

Components of the device 200 are shown in detail in FIG. 2a and FIG. 2b. In FIG. 2a the device 200 consists of at least one sensor, such as but not limited to a microphone, capable of measuring a signal correlated with the tinnitus perceived by the patient. For purposes of this example, the said sensor is depicted as a microphone 201. Note, however that said sensor may be another device mounted within or external to the device 200. The device 200 also contains at least one acoustic source, such as but not limited to the use of at least one ordinary loud speaker as depicted in this example, 202 capable of generating a secondary acoustic field within the ear canal 101. The device 200 also has at least one controller 203 capable of processing the measured signals and computing the signal used to generate the secondary acoustic field. The device 200 may be capable of estimating, computing and/or storing transfer functions and other information relating various locations of the ear to the placement of the device 200 within the ear canal.

FIG. 2b shows an alternative means of obtaining an input to the controller 203. Here, an input signal 204 correlated with the perceived tinnitus but originating from some device other than the microphone 201 is input to the controller 203. The controller 203 then outputs a signal to generate the secondary acoustic field by means of the acoustic source 202. Note that input signal 204 may be due to but not limited to measurements of neurological responses such as functional magnetic resonance imaging (fMRI) or other such measurements.

FIG. 3a and FIG. 3b depict the forward (M 1) and reverse (M2) middle ear pressure gain. As shown schematically in FIG. 3a, the forward (M1) middle ear pressure gain describes the acoustic transfer function between the ear canal and the cochlea given a sound wave originating in the ear canal and propagating towards the cochlea. The reverse (M2) middle ear pressure gain describes the acoustic transfer function between the ear canal and the cochlea given a sound wave originating at the cochlea and propagating towards the ear canal. Note, that the middle ear pressure gain is non-linear and therefore M1 M2.

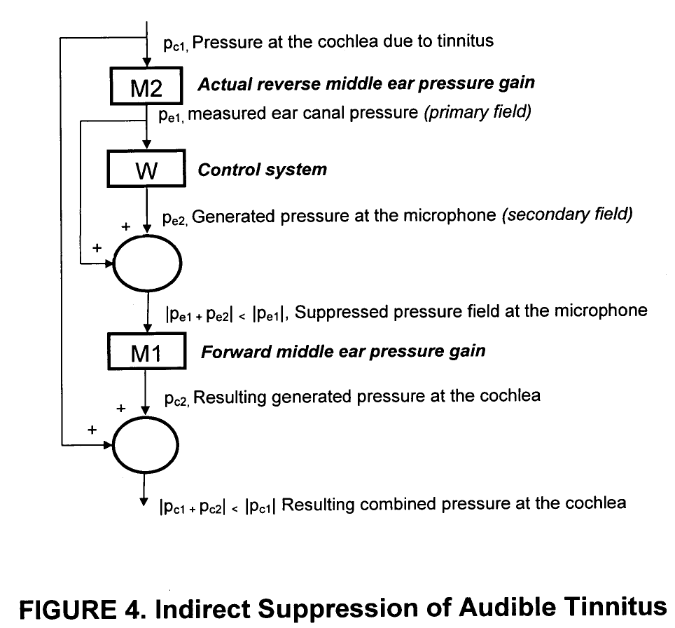

FIG. 4 shows the indirect minimization method which involves suppressing acoustic pressure due to tinnitus in the ear canal at the point of measurement. This control approach requires knowledge in the control system of M1 and M2 or some other acoustic relation between the ear canal at the point of measurement and the cochlea. However, the resulting acoustic pressure at the cochlea may then be estimated knowing the total pressure in the ear canal and the estimated M1 as depicted in the diagram shown in FIG. 3.

Acoustic pressure due to tinnitus is sensed in the ear canal, pₑ₁. This sensed acoustic pressure may then be minimized, at the point of measurement, within the ear canal using a collocated or nearly-collocated sensor/source pair such as but not limited to a microphone and loudspeaker. The control system generates a secondary acoustic pressure, pₑ₂, being of equal frequency content and magnitude and opposing phase relative to the primary acoustic pressure, pₑ₁. The secondary acoustic pressure wave then cancels or suppresses the primary acoustic pressure wave due to the tinnitus such that |pₑ₁+pₑ₂|<|pₑ₁|. The suppression perceived by the patient is then a by-product of the control effort performed to minimize the acoustic pressure due to tinnitus occurring in the ear canal at the point of measurement. This suppression would then be the acoustic pressure at the cochlea due to the tinnitus, p_{c1}, summed with the acoustic pressure resulting at the cochlea p_{c2}, due to the generated acoustic pressure in the ear canal, pₑ₂, convolved with the actual forward middle ear acoustic transfer function, M1, such that |p_{c1}+p_{c2}|<|p_{c1}|. Note, however, for the indirect minimization of audible tinnitus, it is emphasized that the pressures at the cochlea are not considered in the controller, W.

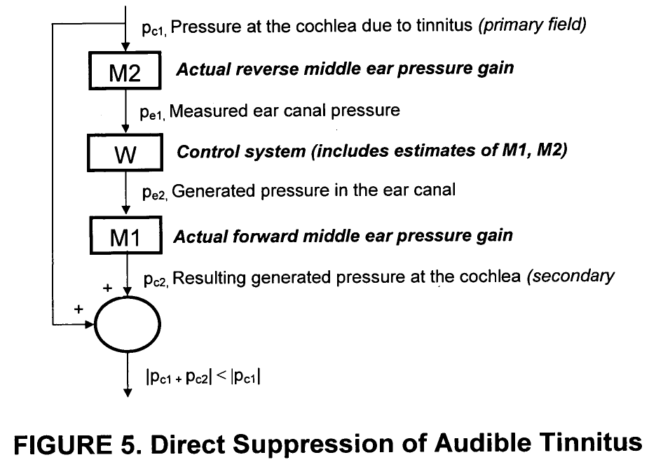

FIG. 5 shows the direct minimization method that also involves the suppression of audible tinnitus but first requires estimating the acoustic relation between the ear canal at or near the point of measurement and the cochlea. Such an estimate may be made using, but not limited to, the middle ear acoustic transfer functions M1 and M2.

The direct minimization method uses a control system having benefit either of previously determined knowledge of the acoustic relation between the ear canal and the cochlea or the ability to estimate such a relation prior to and/or during the control effort. The block diagram for this innovation, as shown using M1 and M2, is depicted in FIG. 4. Acoustic pressure originating at the cochlea, p_{c1}, convolved with the actual reverse middle ear acoustic transfer function, M2, results in an acoustic pressure in the ear canal, pₑ₁, due to the audible tinnitus. The acoustic pressure in the ear canal, pₑ₁, is measured by the device and processed by the controller, W, which estimates p_{c1} and generates a secondary acoustic pressure in the ear canal, pₑ₂ which intends to result in p_{c2} when convolved with the actual forward middle ear acoustic transfer function, M1. The two pressures at the cochlea, p_{c1} and p_{c2}, are of equal magnitude and frequency content but of opposing phase thus suppressing the level of tinnitus perceived by the patient such that |p_{c1}+p_{c2}|<|p_{c1}|.

FIG. 6 shows the case of the minimization of inaudible tinnitus, in which no acoustic pressure may be measured in the ear canal although the patient does indeed perceive a disturbance in the absence of any noise source external to the affected ear. It is conceived that a signal, correlated with the perceived disturbance due to the inaudible tinnitus, may be sensed, either directly or indirectly, by measuring, for instance but not limited to, the activity of the neurological system. Such a measurement may be used to identify the frequency content, magnitude and phase of the perceived sound such that a real, measurable acoustic pressure may be generated in the ear canal. Such a sound may be generated such that it effectively cancels the perceived noise due to the inaudible tinnitus. In other words, an acoustic pressure may be generated to cancel the perceived noise due to the inaudible tinnitus.

A signal, c, correlated with the sound, sₚ₁, perceived by the subject due to the inaudible tinnitus, is sensed. Such a signal, c, may be, but is not limited to be, that of measured neurological activity. The signal, c, may then be input to the controller, W, such that a measurable acoustic pressure, pₑ₂, may then be generated in the ear canal which, via M1, results in an acoustic pressure existing at the cochlea, p_{c2}. This real, measurable acoustic pressure existing in the cochlea may then be perceived as, sₚ₂, by the subject being of equal magnitude and frequency content yet of opposite phase to that of the perceived signal due to the inaudible tinnitus, sₚ₁. Therefore, the two perceived sounds—that due to the inaudible tinnitus and that due to the acoustic pressure generated by the controller—then cancel resulting in a perceived suppression of the inaudible tinnitus such that |Sₚ₁+Sₚ₂|<|Sₚ₁|.

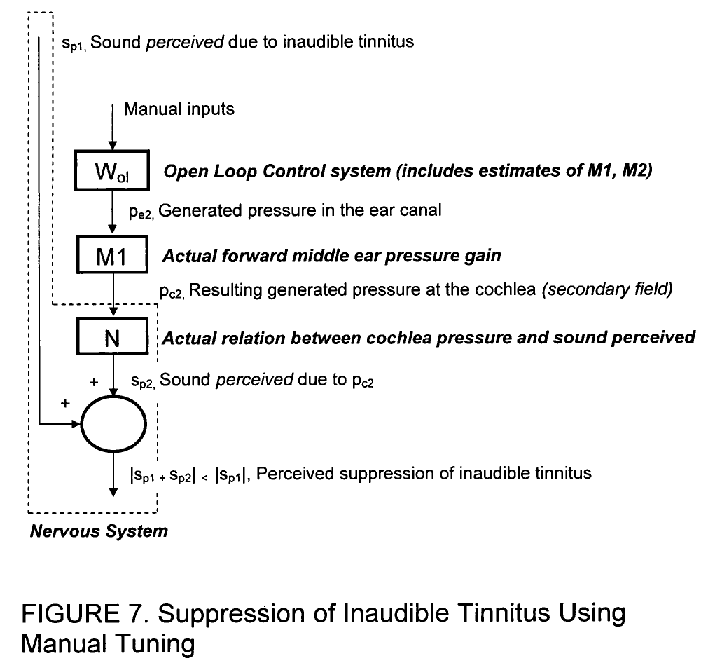

FIG. 7 shows the suppression of inaudible tinnitus using manual tuning. This process is similar to that described in FIG. 6 without the benefit of measuring a signal correlated with the perceived tinnitus and therefore no knowledge of the inaudible tinnitus is available. Suppression is achieved by generating such a pressure and iteratively varying the frequency content and phase until the patient perceives a reduction of the disturbance.

The invention has been described in terms of the several embodiments. It is to be understood that the preceding description is given to illustrate various embodiments of the present inventive concepts. The specific examples are not to be considered as limiting, except in accordance with the following claims.

1. An apparatus for suppressing tinnitus comprising:
a power source;
at least one sensor for measuring at least one signal correlated with a perceived sound electrically connected to the power source having a sensor output;
a filtering component applied to said sensor output wherein said filtering component has a filtered output that reduces perceived tinnitus;
an amplification component that amplifies said filtered output; and
at least one acoustic source electrically connected to said amplification component producing an acoustic wave sufficient to reduce perceived tinnitus.

2. The apparatus of claim 1 further comprising:
at least one control system capable of reading the at least one signal; and
a circuit or processor for producing computations sufficiently fast to generate the secondary acoustic field used to cancel the real or perceived sound due to the tinnitus.

3. The apparatus of claim 1 wherein the sensor is a microphone.

4. The apparatus of claim 1 wherein the acoustic source is a speaker.

5. The apparatus of claim 1 wherein the source of power is at least one battery.

6. The apparatus of claim 1 wherein the circuit is a computer.

7. The method of claim 1, for the indirect suppression of audible tinnitus, comprising the steps of:
the pressure at the cochlea occurring due to tinnitus; and
then resulting, via the actual reverse middle ear pressure gain, M2 in the ear canal as a pressure (primary field) pₑ₁ due to the audible tinnitus where it is measured by a microphone; and
inputting pₑ₁ into the controller W to perform the necessary computations such that the secondary field pₑ₂ may be generated; and
generating the secondary field pₑ₂ at the microphone placed within the ear canal such that the sum of the primary and secondary fields—the suppressed ear canal field—results in a lesser magnitude within the ear canal than that due to pₑ₁ alone; and
the acoustic energy due to the secondary field generated within the ear canal, pₑ₂ occurs, via the real forward middle ear pressure gain, M1 as a pressure at the cochlea, p_{c2} combining with the pressure at the cochlea due to the tinnitus, p_{c1} such that the loudness of the tinnitus is suppressed.

8. The method of claim 1, for the direct suppression of audible tinnitus, comprising the steps of:
the pressure at the cochlea occurring due to tinnitus; and
then resulting, via the actual reverse middle ear pressure gain, M2 in the ear canal as a pressure (primary field) pₑ₁ due to the audible tinnitus where it is measured by at least one microphone; and
inputting pₑ₁ into the at least one controller W; and
predicting the pressure at the cochlea due to tinnitus, pc1 using an estimate of the forward middle ear pressure gain; and
computing the required generated pressure at the cochlea p_{c2} required to suppress p_{c1}; and
predicting the required generated acoustic pressure within the ear canal, pₑ₂ using estimates of the forward middle ear pressure gain such that when the acoustic energy due to pₑ₂ travels to the cochlea via the real forward middle ear pressure gain M1, the generated pressure at the cochlea p_{c2} suppresses the pressure due to tinnitus, p_{c1}; and
generating the secondary pressure pₑ₂ within the ear canal which then travels to the cochlea via the actual forward middle ear pressure gain M1 resulting in the actual generated pressure at the cochlea, p_{c2} suppressing the pressure at the cochlea due to tinnitus, p_{c1}.

9. The method of claim 1, for the suppression of inaudible tinnitus, comprising the steps of:
a sound perceived by the patient due to inaudible tinnitus Sₚ₁; and
the at least one measured signal c correlated with the inaudible tinnitus Sₚ₁; and
the at least one control system W used to generate a pressure within the ear canal pₑ₂ resulting, via the real forward middle ear pressure gain in a pressure at the cochlea p_{c2}; and
the perceived sound Sₚ₂ due to the actual relation N between the cochlea pressure and the perceived sound such that when summed, the sound perceived due to the inaudible tinnitus, Sp1 is suppressed.

10. The method of claim 1, for the suppression of inaudible tinnitus using manual tuning, comprising the steps of:
a sound perceived by the patient due to inaudible tinnitus Sₚ₁; and
an open loop controller Wₒₗ having manual inputs for adjusting control parameters such that a pressure may be generated in the ear canal pₑ₂; and
resulting in a pressure at the cochlea p_{c2} via the actual forward middle ear pressure gain M1; and
the perceived sound Sₚ₂ due to the actual relation N between the cochlea pressure and the perceived sound such that when summed, the sound perceived due to the inaudible tinnitus, Sp1 is suppressed.

11. A method of suppressing sound due to tinnitus comprising the steps of:
providing at least one sensor for measuring at least one signal correlated with sound;
providing at least one acoustic source;
providing a source of power;
providing filtering components;
providing amplification components;
measuring a signal correlated with sound heard;
computing the control output signal; and
generating the control sound field used for suppressing the tinnitus or otoacoustic emission.

12. The method of claim 11 further comprising the steps of:
providing a patient subjected to the presence of tinnitus heard or perceived in either one or both ears; and
placing the apparatus for suppressing tinnitus into the patient's ear canal.

13. The apparatus of claim 1 further comprising:
at least one control system capable of reading the at least one signal correlated with the sound either heard or perceived by the patient; and
the use of the estimated acoustic relationship between the ear canal and the cochlea such as but not limited to the use of the forward and reverse middle ear pressure gains denoted herein as M1 and M2, respectively; and
a controller, being either analog or digital or of a combined analog/digital design, used for performing computations sufficiently fast to generate the secondary acoustic field used to cancel the real or perceived sound due to the tinnitus.
